(19) United States
(12) Patent Application Publication
(10) Pub. No.: US 2012/0283434 A1
(43) Pub. Date: Nov. 8, 2012

(54) PROCESS FOR THE PREPARATION OF RIVAROXABAN AND INTERMEDIATES THEREOF

(75) Inventors: Llorenç Rafecas Jané, Barcelona (ES); Alexander Christian Comely, Barcelona (ES); Alessandro Ferrali, Barcelona (ES); Celia Amela Cortés, Barcelona (ES); Mireia Pastó Aguilà, Barcelona (ES)

(73) Assignee: ENANTIA, S.L., Barcelona (ES)

(21) Appl. No.: 13/520,337

(22) PCT Filed: Jan. 3, 2011
(86) PCT No.: PCT/EP11/50003

§ 371 Date: Jul. 2, 2012

(30) Foreign Application Priority Data
Jan. 4, 2010 (EP) EP 10382001.5

(51) Int. Cl. C07D 413/14; C07D 413/10

(52) U.S. Cl. 544/137

(57) ABSTRACT

A process for the preparation of rivaroxaban, or a pharmaceutically acceptable salt thereof, or a solvate thereof, including a hydrate, comprising submitting an amine compound of formula (III) wherein R₁ is a (C₄-C₁₀)-alkyl radical which is attached to the N atom by a tertiary C atom, first to an acylation reaction and then to a dealkylation reaction.

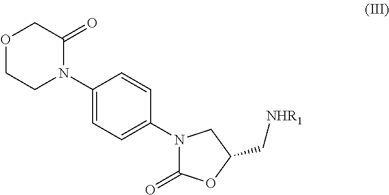

The present invention relates to a process for the preparation of rivaroxaban, as well as to some new intermediates useful in such a preparation process.

### BACKGROUND ART

Rivaroxaban is the International Non-proprietary Name (INN) of 5-chloro-N-{[(5S)-2-oxo-3-[4-(3-oxomorpholin-4-yl)phenyl]oxazolidin-5-yl]methyl}thiophene-2-carboxamide, available under the brand name Xarelto®, property of Bayer, in 10 mg tablets. The Registry number is CAS No. 366789-02-8. Rivaroxaban is currently used as anti-thrombotic agent.

The structure of rivaroxaban corresponds to formula (I):

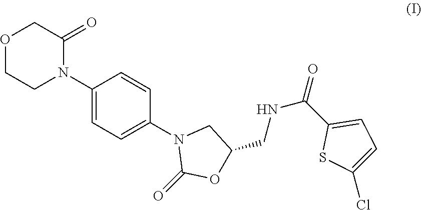

Rivaroxaban was first disclosed in patent EP 1261606. The synthesis of rivaroxaban is proposed by reaction between 4-(4-aminophenyl)morpholin-3-one and a terminal epoxide such as 2-((oxiran-2-yl)methyl)isoindoline-1,3-dione or 5-chloro-N-((oxiran-2-yl)methyl)thiophene-2-carboxamide with subsequent formation of the oxazolidinone. The preparation of 4-(4-((S)-5-(aminomethyl)-2-oxooxazolidin-3-yl)phenyl)morpholin-3-one is referred to S. J. Brickner et al., J. Med. Chem.—1996, Vol. 39, pp. 673, wherein an alcohol precursor is first transformed to a mesylate which is then transformed into an amine. The transformation of the mesylate into the amine can be carried out through the formation of the corresponding azide followed by a reduction reaction. Example 2 of patent EP 1261606, which specifically discloses the synthesis of rivaroxaban, gives no yield for the reaction. Another way to transform the mesylate is through the displacement of the sulfonate group with a phthalimide salt, i.e. potassium phthalimide, followed by a deprotection reaction or, alternatively, by direct reaction with ammonium hydroxide. The reaction with a phthalimide salt is also disclosed in patent application WO 2003/2556. The reaction with ammonium hydroxide can be seen in the patent EP 1114819, wherein a 5-aminomethyl substituted oxazolidinone amine is prepared by contacting oxazolidinone sulfonates with ammonium hydroxide in a sealed system at a pressure of 270 kPa. Finally, the last step is the acylation of the amine thus obtained.

All these alternative reactions to yield rivaroxaban present drawbacks which should be solved. The main drawbacks of using an azide, such as sodium azide, are that it constitutes a dangerous route due to its explosive capacity.

When the sulfonate compound is reacted with aqueous ammonia, high temperatures and high pressure are required and, therefore, this process cannot be performed in ordinary reactors but in special reactors adapted for high pressure. Finally, the reaction sequence which involves the formation of the sulfonate and its reaction with potassium phthalimide produces by-products which are difficult to separate from the desired product.

The wide prevalence of illnesses related to blood coagulation makes rivaroxaban an important active ingredient. As exposed before, to date the field provides hazardous, multi-step, and complex methods for obtaining rivaroxaban. Thus, synthetic approaches solving the disadvantages of the known methods are of special interest while providing rivaroxaban in high yields.

### SUMMARY OF THE INVENTION

The inventors have found a new preparation process for rivaroxaban from new intermediate compounds, which proceeds with good yields while achieving a good chemical and optical purity. The new process comprises the activation of an alcohol precursor of rivaroxaban, amination with some specific amines bearing an alkyl, which may be removed, and the submission of the compound obtained to an acetylation reaction and to a dealkylation reaction. The herewith proposed process of the present invention is particularly advantageous in its practical industrial application since it is much more cost effective. No chromatography is required and final rivaroxaban is obtained with high purity (by HPLC up to 98%). Additionally the last two steps of the process may be performed in one pot. The process represents a safer route to yield rivaroxaban, and has minimum environmental impact. All these advantages allow for advantageous scale up.

For the transformation of the activated alcohol (a sulfonated compound) to the amine, the process of the present invention is advantageous since it avoids some of the drawbacks of the previously described processes. Indeed, when phthalimide is used instead of the amines of the present invention, while being an expensive reactive, there are difficulties due to the obtention of side-products difficult to be isolated from the desired product. The deprotection reaction of phthalimide compounds can be carried out with hydrazine or using basic conditions. As known in the art, the use of hydrazine involves several process difficulties and the basic conditions require high temperatures which may not be compatible with rivaroxaban.

On the other hand, if ammonia is used instead of the specific amines of the present invention, a very large excess of this reagent must be used. This has an important environmental impact and yields are low because of the presence of side-products such as secondary amines. All these aspects make application of the route with ammonia difficult at industrial scale.

The process of the present invention presents a different approach to the known processes for obtaining rivaroxaban or its salts. It is based on the introduction of a substituted amino group by an amination reaction, which is a key step of the process, and which has several advantages mainly in terms of yields, economy and environmental impact.

The rivaroxaban obtained following the process of the present invention is of high purity with respect to the R-enantiomer and other impurities, without the need of tedious complicated purification steps such as chromatography. This is due to the obtention of pure intermediates, in particular the intermediate compound of formula (III) of the process of the present invention. Intermediate of formula (III) can be purified by crystallization or by formation of salts.

Thus, by the process of the present invention, rivaroxaban can be produced safely and simply in high yield.

Accordingly, an aspect of the present invention is to provide a process for the preparation of rivaroxaban of formula (I), or its pharmaceutically acceptable salts

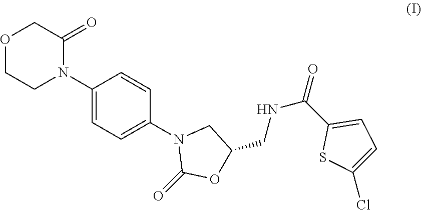

comprising submitting a compound of formula (III) as defined below, first to an acylation reaction with a compound of formula (IV),

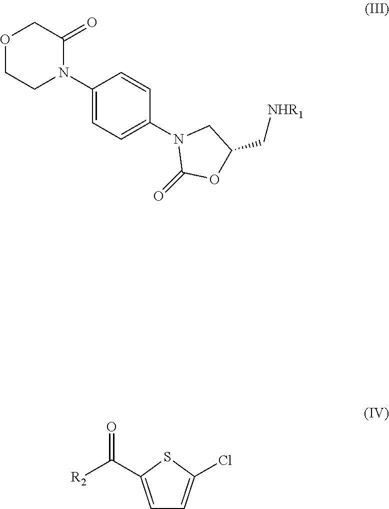

and then to a dealkylation reaction; and, optionally, treating the compound (I) thus obtained with a pharmaceutically acceptable acid to form the corresponding pharmaceutically acceptable salt.

In compound (III), R₁ is a (C₄-C₁₀)-alkyl radical which is attached to the N atom by a tertiary C atom. A (C₄-C₁₀)-alkyl radical may be removed from the N atom to which it is attached by a dealkylation reaction.

In compound (IV) R₂ is a radical selected from the group consisting of a halogen and a radical of formula (C₁-C₈)COO.

Another aspect of the present invention is to provide intermediate compounds which are useful in the preparation process described above. Thus, compounds of formula (III) as defined above are new and are also part of the invention.

Compounds of formula (III) are key intermediates of the process which are obtained with good yields and good chemical and optical purity. These compounds might be purified by crystallization or by formation of salts such as the methansulfonate salt. The Examples included in this document illustrate the results obtained regarding purity and yield of these intermediates.

Compounds of formula (II), which are obtained when compounds of formula (III) are submitted to an acylation reaction, are new and also form part of the invention.

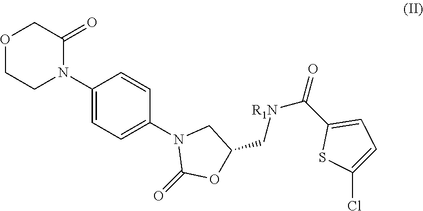

In formula (II), R₁ is a (C₄-C₁₀)-alkyl radical which is attached to the N atom by a tertiary C atom.

Some related compounds similar to those of formula (II) are disclosed by Roehring et al., in “Discovery of the Novel Antithrombotic Agent 5-Chloro-N-({(5S)-2-oxo-3-[4-(3-oxomorpholin-4-yl)phenyl]-1,3-oxazolidin-5-yl}methyl)thiophene-2-carboxamide (BAY 59-7939): An Oral, Direct Factor Xa Inhibitor”, Journal of Medicinal Chemistry—2005, Vol. 48, pp. 5900. Roehring discloses, among others, a compound of formula (F)

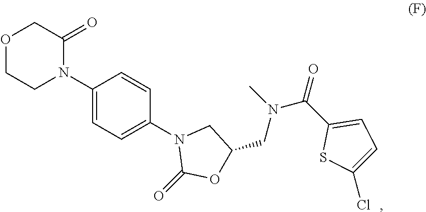

in which the amino group is substituted by a methyl radical (or what is the same, and according to the nomenclature of the present invention, R₁ is methyl) (Example 27 of Table 2 in Roehring et al., supra). However, this compound is not useful as intermediate for the preparation of rivaroxaban, since this compound cannot be dealkylated under the conditions described herein.

Compounds of formula (II) are also key intermediates of the process which are obtained with good yields and good chemical and optical purity. The Examples included in this document illustrate the results obtained regarding purity and yield of these intermediates.

Advantageously, compounds of formula (II) might be isolated or directly transformed into rivaroxaban in a one pot process.

### DETAILED DESCRIPTION OF THE INVENTION

As mentioned above, the process of the present invention comprises submitting a compound (III) to an acylation reaction and to a dealkylation reaction.

The acylation reaction of compound (III) as defined above gives rise to the intermediate compound of formula (II) where R₁ is a (C₄-C₁₀)-alkyl radical which is attached to the N atom by a tertiary C atom.

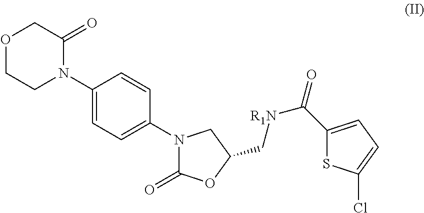

The intermediate compounds of formula (II), being key intermediates of the process, are obtained with good yields and good chemical and optical purity.

As above exposed, when, in the compound of formula (III), R₁ is a (C₄-C₁₀)-alkyl radical which is attached to the N atom by a tertiary C atom, such as a tert-(C₄-C₈)alkyl radical, namely, tert-butyl or tert-octyl, the sequence of reactions to obtain rivaroxaban is first an acylation reaction and then a dealkylation reaction. Thus, in a preferred embodiment, the acylation reaction gives rise to compounds of formula (II) where R₁ is tert-(C₄-C₈)alkyl, namely tert-butyl or tert-octyl.

The acylation reaction of compound (III) to yield compound (II) can be carried out in an appropriate solvent, and in the presence of a base, using either a compound of formula (IV), wherein R₂ is a halogen, preferably a chloride, or a (C₁-C₈)COO radical, this latter radical representing an anhydride of the compound of formula (IV). Appropriate solvents for carrying out the acylation include chlorine containing solvents such as dichloromethane, (C₂-C₈)-ethers such as tert-butyl methyl ether, (C₃-C₇)-ketones such as methylisobutylketone, (C₆-C₈)-aromatic hydrocarbons such as toluene or xylene, or ethyl acetate. Suitable bases include pyridine or tertiary alkyl amines such as triethylamine. Generally, the reaction is carried out at a temperature comprised between room temperature and approximately 120° C. depending on the reagent used. If a halide of the compound of formula (IV) is used, generally the reaction is carried out at a temperature comprised between room temperature and 120° C. If an anhydride of formula (IV) is used, then generally the reaction is carried out at high temperature within the specified range.

The dealkylation reaction of the compound of formula (II) to yield the compound of formula (I), where R₁ is a (C₄-C₁₀)-alkyl radical which is attached to the N atom by a tertiary C atom, can be carried out using a solution of hydrogen chloride or trifluoroacetic acid in an appropriate solvent. Examples of appropriate solvents include chlorine containing solvents such as dichloromethane, ethers such as tert-butyl methyl ether, dioxane or tetrahydrofuran, (C₃-C₇)-ketones such as methylisobutylketone, (C₆-C₈)-aromatic hydrocarbons such as toluene or xylene, ethyl acetate, or water.

These two steps acylation and dealkylation might be performed in one pot process. This has the additional advantage that a simplified process avoiding purifications and isolation steps is carried out.

Rivaroxaban obtained by the process of the present invention may be converted into pharmaceutically acceptable salts by known methods described in the art, for instance, by the reaction of rivaroxaban free base with a sufficient amount of a pharmaceutically acceptable acid to yield the corresponding salt.

Rivaroxaban is a compound with a low solubility in most solvents. This fact represents a drawback for the purification of the same. The inventors have further found that rivaroxaban may be obtained at high yields and purity levels if once synthesized it is recrystallized from a solvent of the group consisting of dioxane, a mixture of (C₁-C₄)-alcohol/water, a mixture of (C₂-C₈)-ethers and water, and a mixture of (C₂-C₈)-ethers and (C₁-C₄)-alcohols, such as dioxane in (C₁-C₄)-alcohol. A preferred mixture of (C₁-C₄)-alcohol/water is ethanol/water.

The term “pharmaceutically acceptable salts” used herein encompasses any salt formed from organic and inorganic acids. Examples of inorganic acids include hydrochloric acid, hydrobromic acid, hydroiodic acid, nitric acid, perchloric acid, sulfuric acid or phosphoric acid. Examples of organic acids include methanesulfonic acid, trifluoromethanesulfonic acid, ethanesulfonic acid, 2-hydroxyethanesulfonic acid, benzenesulfonic acid, p-toluenesulfonic acid, fumaric acid, citric acid, oxalic acid, acetic acid and malic acid. There is no limitation regarding the salts except that, if used for therapeutic purposes, they must be pharmaceutically acceptable.

Compound of formula (I) or its salts can exist in solvated, as well as unsolvated forms, including hydrated forms, ie. the rivaroxaban can contain in its structure stoichiometric amounts of solvent in the case of solvates, or of water in the case of hydrates. It is to be understood that the invention encompasses all such solvated, as well as unsolvated forms. The obtention of solvates and hydrates depends on the solvent used and the crystallization conditions that can be determined by the skilled person.

The intermediate compound of formula (III) as defined above can be obtained by submitting a compound of formula (V), wherein R₃ is a radical selected from the group consisting of (C₁-C₄)-alkyl, phenyl, and phenyl mono- or disubstituted by a (C₁-C₄)-alkyl radical to an amination reaction. The amination reaction is carried out by reacting said compound (V) with an amine of formula R₁NH₂ where R₁ is a (C₄-C₁₀)-alkyl radical which is attached to the N atom by a tertiary C atom.

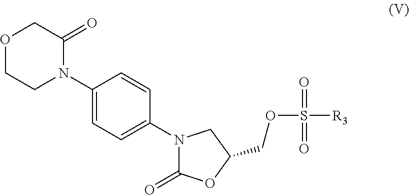

Preferred sulfonate compounds of formula (V) for use in the process of the present invention are those where the sulfonate (V) is a mesylate (R₃=methyl), a besylate (R₃=phenyl) or a tosylate (R₃=4-methylphenyl). The most preferred sulfonate (V) is the mesylate.

In a preferred embodiment, the amine used in the amination reaction is a tert-(C₄-C₈)alkyl amine, preferably a tert-butylamine or tert-octylamine. Appropriate solvents for carrying out the amination reaction include chlorine containing solvents such as dichloromethane, ethers such as 2-methyltetrahydrofuran, (C₆-C₈)-aromatic hydrocarbons such as toluene and xylene, acetates such as ethyl acetate, amides such as DMF and (C₁-C₆)-alcohols

In another preferred embodiment, the amination reaction is carried out using a (C₁-C₆)-alcohol as solvent. More preferably, the alcohol used is isopropanol.

The intermediate compounds of formula (III), being a key intermediates of the process, are obtained with good yields and good chemical and optical purity.

In a preferred embodiment the compounds of formula (III) are obtained with an improved chemical and optical purity by carrying out the amination reaction and further performing a crystallization step in a solvent selected from the group consisting of (C₆-C₈)-aromatic hydrocarbons such as toluene or xylene, (C₂-C₈)-ethers such as tetrahydrofuran, (C₃-C₇)-ketones such as methylisobutylketone, (C₁-C₄)-alkyl acetates as ethyl acetate, and alcohols as isobutanol.

The most preferred solvent to be used is methylisobutylketone

Yet in another preferred embodiment the purity of the compound of formula (III) is highly improved if further to the amination reaction the formation of a salt of the compound of formula (III) is carried out by reacting the corresponding compound of formula (III) with an appropriate acid in a suitable solvent. Preferred salts include those selected from the group consisting of sulfonate, hydrochloride, hydrobromide, and tosylate salts.

The Examples included in this document illustrate the results obtained regarding purity and yield of these intermediates. In particular, Example 9 illustrates the crystallization of 4-(4-((S)-5-(tert-butylaminomethyl)-2-oxooxazolidin-3-yl)phenyl)morpholin-3-one (compound III with R₁=tert-butyl), wherein the compound is finally obtained with a purity of 93% by HPLC-MS. Or in Example 8 this compound is obtained passing through the corresponding methansulfonate salt with a purity of 97% by HPLC-MS.

In another preferred embodiment, the sulfonate of formula (V) can be prepared from the corresponding alcohol of formula (VII) by reaction with the corresponding sulfonyl halide of formula X—SO₂—R₃, where R₃ has the same meaning defined above for compound (V), and X represents any halogen atom, preferably chloride.

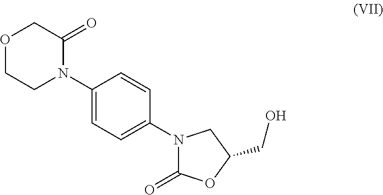

This reaction can be carried out in an appropriate solvent and in the presence of a tertiary amine, at a temperature comprised between 0° C. and 70° C., preferably at a temperature comprised between 0° C. and room temperature. More preferably, the reaction is carried out at low temperatures. Common solvents for this reaction include chlorine-containing solvents such as methylene chloride or 1,2-dichloroethane, (C₆-C₈)-aromatic hydrocarbons such as toluene or xylene, (C₂-C₈)-ethers such as tetrahydrofuran, (C₃-C₇)-ketones such as methylethylketone, and dimethylformamide. Examples of suitable tertiary amines are diisopropylethylamine and triethylamine.

The starting alcohol of formula (VII) is commercial and can be prepared by any of the methods known in the art. Preferred methods to obtain the alcohol of formula (VII) include those mentioned by Roehring et al. in “Discovery of the Novel Antithrombotic Agent 5-Chloro-N-({(5S)-2-oxo-3-[4-(3-oxomorpholin-4-yl)phenyl]-1,3-oxazolidin-5-yl}methyl)thiophene-2-carboxamide (BAY 59-7939): An Oral, Direct Factor Xa Inhibitor”, J. Med. Chem—2005, Vol. 48, pp. 5900-5908. In this document the alcohol of formula (VII) is obtained from a compound of formula (VIII), wherein Cbz is the protective group carboxybenzyl, which is converted to the alcohol with (R)-(−)-glycidol butyrate of formula (IX) below, in the presence of n-butyllithium or lithium tert-butoxide in an appropriate solvent, such us tetrahydrofuran.

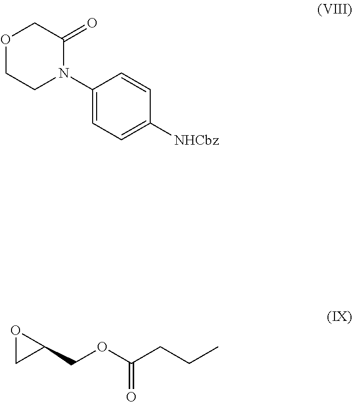

The skilled man in the art will easily deduce that compound of formula (VIII) is the direct compound obtainable when the corresponding amine (aniline) without the protective group is submitted to the known reaction of protection of amine groups. Thus, a compound of formula (X) is reacted with carboxybenzyl chloride (CbzCl) in the presence of a base, and in an appropriate solvent. Suitable bases include hydrogen carbonate salts, such as sodium hydrogen carbonate. Appropriate solvents include mixtures of water with aprotic solvents, such as (C₃-C₈)-ketones, namely acetone.

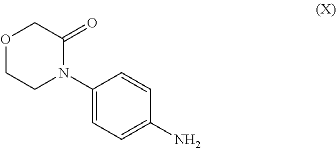

This compound of formula (X) is commercial or can be prepared from the corresponding nitro radical containing compound of formula (XI) by means of a reduction reaction with hydrogen gas and in the presence of palladium(0)/carbon. The reaction is usually carried out in tetrahydrofuran.

Alternatively, the reaction can be carried out using an alcohol as solvent, preferably methanol or ethanol according to DE 10342570.

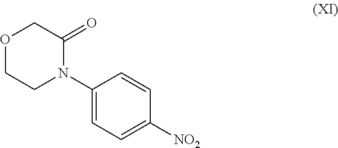

The inventors found that the conversion of compound of formula (XI) to compound of formula (X) can be carried out with high yields using wet palladium(0)/carbon; or a solvent system which is a mixture of water and a solvent selected from the group consisting of ethers such as tert-butyl methyl ether or tetrahydrofuran, (C₁-C₄)-alcohols such as methanol or ethanol, or ethyl acetate. In particular, use of 10% Pd/C (approximately 50% wet with water) in THF results in complete conversion and a 89% yield of compound (X) (Example 3)

As will be illustrated below in the Examples, the nitro radical containing compound of formula (XI) is commercially available or can be prepared by any of the methods known in the art. Specific examples of synthesis are disclosed in the patent applications WO 2005/26135 and WO 2006/116713.

Throughout the description and claims the word “comprise” and variations of the word, are not intended to exclude other technical features, additives, components, or steps.

The following examples are provided by way of illustration, and they are not intended to be limiting of the present invention.

Purity-related data of the compounds are derived from a HPLC analysis.

Conditions of the HPLC-MS-MS analysis:
Flux: 2 mL/min
Column: Xbridge C18
Phases: Phase A: aqueous solution of NH₄HCO₃ pH 8 Phase B: acetonitrile (ACN)
Detection wavelength: 254 nm
Gradient: From 5 to 100% of solution B in 7 minutes.

Conditions of the HPLC analysis:
Flux: 1 mL/min
Column: Luna C18
Phases: Phase A: aqueous solution 01% HCOOH Phase B: acetonitrile (ACN)
Detection wavelength: 254 nm
Gradient: From 2 to 95% of solution B in 30 minutes.

### EXAMPLES

### Example 1

### Preparation of 4-phenylmorpholin-3-one

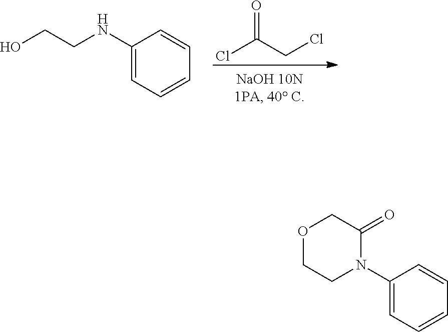

To a solution of 2-anilino ethanol (6.0 mL, 47.8 mmol) in IPA (6 mL) heated to 40° C. were simultaneously added dropwise chloroacetyl chloride (11.4 mL, 143.4 mmol) and 10 N NaOH (29.6 mL, 296 mmol) maintaining the pH around 7-8. After the addition, the mixture was stirred at 40° C. for 10 min, was cooled to 0° C. and was stirred at this temperature for 1 h. The white solid formed was collected by filtration, washed with cold water and dried, affording 4-phenylmorpholin-3-one (5.27 g, 62%) as a white solid. ¹H NMR (400 MHz, CDCl₃) δ 7.42 (t, J=8.0 Hz, 2H), 7.34-7.27 (m, 3H), 4.35 (s, 2H), 4.04 (t, J=5.2 Hz, 2H), 3.77 (t, J=5.2 Hz, 2H).

### Example 2

### Preparation of 4-(4-nitrophenyl)morpholin-3-one (compound XI)

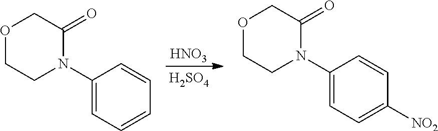

To a solution of 4-phenylmorpholin-3-one (19.9 g, 0.11 mol) in 95% H₂SO₄ (46.6 mL) cooled to −10° C. was carefully added dropwise during 45 min 65% HNO₃ (8 mL). The reddish solution obtained was stirred at this temperature for 1 h and H₂O (110 mL) was added. The mixture was neutralized with 35% NH₄OH until pH=7. The resulting precipitate was filtered, washed with cold water and dried, affording 4-(4-nitrophenyl)morpholin-3-one (or compound XI) (20 g, 83%) as a brown solid.

¹H NMR (200 MHz, CDCl₃) δ 8.27 (d, J=9.4 Hz, 2H), 7.62 (d, J=9.4 Hz, 2 H), 4.38 (s, 2H), 4.08 (t, J=5.0 Hz, 2H), 3.85 (t, J=5.0 Hz, 2H).

### Example 3

### Preparation of 4-(4-aminophenyl)morpholin-3-one (compound X)

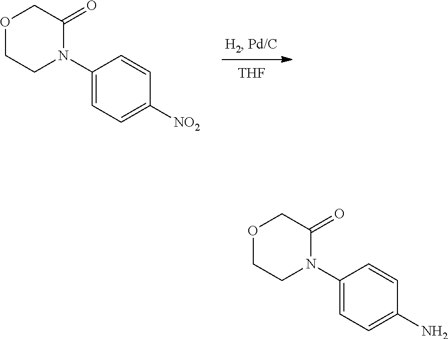

To a solution of 4-(4-nitrophenyl)morpholin-3-one (19.9 g, 89.5 mmol) in THF (200 mL) was added Pd/C (5%) (1.7 g, water content: 58%). The reactor was filled with H₂ and the mixture was stirred at 70° C. under a pressure of 3 bars during 6.5 h. As the reaction was not complete (by HPLC-MS) more Pd/C was added (900 mg) and the mixture was stirred under the same reaction conditions until all starting material was consumed. The catalyst was filtered off through celite, and the filtrate was concentrated to afford 4-(4-aminophenyl)morpholin-3-one (or compound of formula X) (15.3 g, 89%) as a brown solid. ¹H NMR (400 MHz, CDCl₃) δ 7.07 (d, J=8.8 Hz, 2H), 6.69 (d, J=8.8 Hz, 2H), 4.32 (s, 2H), 4.00 (t, J=4.8 Hz, 2H), 3.72 (bs, 2H), 3.69 (t, J=4.8 Hz, 2H).

### Example 4

### Preparation of benzyl 4-(3-oxomorpholino)phenylcarbamate (compound VIII)

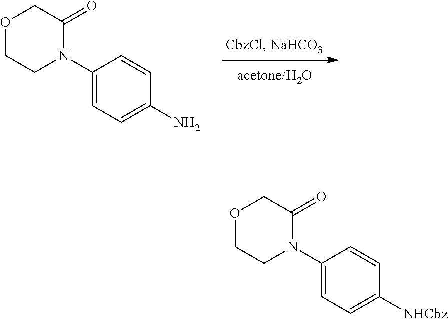

To a solution of 4-(4-aminophenyl)morpholin-3-one (3.73 g, 19.40 mmol) in acetone (80 mL) were added H₂O (40 mL) and NaHCO₃ (3.26 g, 38.80 mmol). The mixture was cooled to 0° C. and CbzCl (2.90 mL, 20.60 mmol) was added dropwise. The mixture was stirred at room temperature for 4 h and poured onto ice/H₂O. The precipitate was filtered, washed with H₂O and hexane and dried, affording benzyl 4-(3-oxomorpholino)phenylcarbamate (or compound VIII) (5.44 g, 86%) as a white solid. ¹H NMR (200 MHz, CDCl₃) δ 7.44-7.36 (m, 7H), 7.25-7.22 (m, 2H), 6.86 (bs, 1H), 5.20 (s, 2H), 4.32 (s, 2H), 4.01 (t, J=5.4 Hz, 2H), 3.72 (t, J=5.4 Hz, 2H).

### Example 5

### Preparation of 4-(4-((R)-5-(hydroxymethyl)-2-oxooxazolidin-3-yl)phenyl)morpholin-3-one (compound VII)

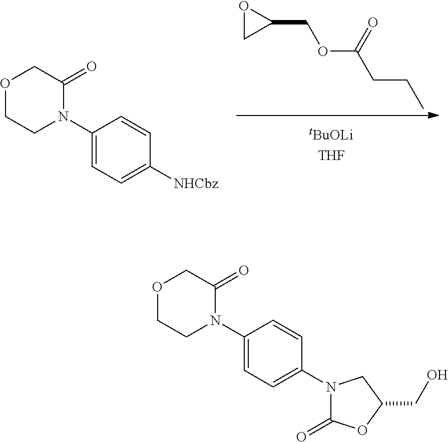

To a solution of benzyl 4-(3-oxomorpholino)phenylcarbamate (3.00 g, 9.20 mmol) in anhydrous THF (28 mL) was added dropwise a solution of ^{t}BuOLi in THF (1.0 M, 18.4 mL, 18.4 mmol). (R)-Glycidyl butyrate (1.43 mL, 10.1 mmol) was added and the mixture was stirred at room temperature for 24 h. After dilution with a saturated NH₄Cl solution (20 mL), THF was evaporated under reduced pressure and the aqueous phase was extracted with CH₂Cl₂ (8×20 mL). The combined organic phases were dried over MgSO₄ and concentrated in vacuo. The crude obtained was suspended in EtOAc (10 mL). The suspension was heated to reflux temperature for 30 min, was cooled to room temperature and was filtered. The filtrate was washed with cold EtOAc, affording 4-(4-((R)-5-(hydroxymethyl)-2-oxooxazolidin-3-yl)phenyl)morpholin-3-one (or compound VII) (2.139 g, 80%) as a brownish solid.

¹H NMR (400 MHz, CDCl₃) δ 7.59 (d, J=9.2 Hz, 2H), 7.34 (d, J=9.2 Hz, 2H), 4.75-4.70 (m, 1H), 4.34 (s, 2H), 4.05-3.93 (m, 5H), 3.76-3.71 (m, 3H).

### Example 6

### Preparation of 4-(4-((R)-5-(methanesulfonyloxymethyl)-2-oxooxazolidin-3-yl)phenyl)morpholin-3-one (compound V)

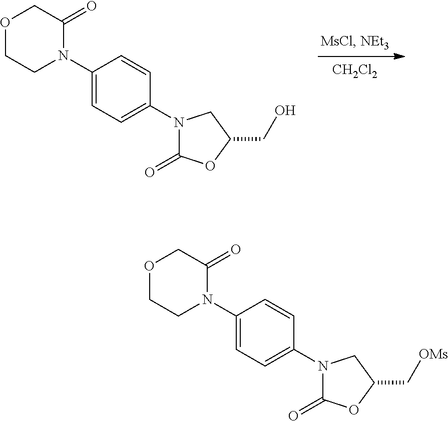

To a solution of 4-(4-((R)-5-(hydroxymethyl)-2-oxooxazolidin-3-yl)phenyl)morpholin-3-one (1.828 g, 6.25 mmol) in anhydrous CH₂Cl₂ (110 mL) cooled to 0° C. were added NEt₃ (2.6 mL, 18.7 mmol) and subsequently MsCl (0.63 mL, 8.12 mmol). The mixture was stirred at room temperature for 4 h. After washing with H₂O and brine, the organic phase was dried over MgSO₄ and was concentrated in vacuo, affording 4-(4-((R)-5-(methanesulfonyloxymethyl)-2-oxooxazolidin-3-yl)phenyl)morpholin-3-one (or compound V) (2.255 g, 97%) as a white solid. ¹H NMR (400 MHz, CDCl₃) δ 7.59 (d, J=9.2 Hz, 2H), 7.37 (d, J=9.2 Hz, 2H), 4.96-4.90 (m, 1H), 4.50 (dd, J=11.6 Hz, 4.0 Hz, 1H), 4.43 (dd, J=11.6 Hz, 4.0 Hz, 1H), 4.34 (s, 2H), 4.17 (dd, J=9.2 Hz, 9.2 Hz, 1H), 4.04 (t, J=4.8 Hz, 2H), 3.98 (dd, J=8.8 Hz, 6.0 Hz, 1H), 3.76 (t, J=5.2 Hz, 2H), 3.11 (s, 3H).

### Example 7

### Preparation of 4-(4-((S)-5-(tert-butylaminomethyl)-2-oxooxazolidin-3-yl)phenyl)morpholin-3-one (compound III with R₁=tert-butyl) in IPA

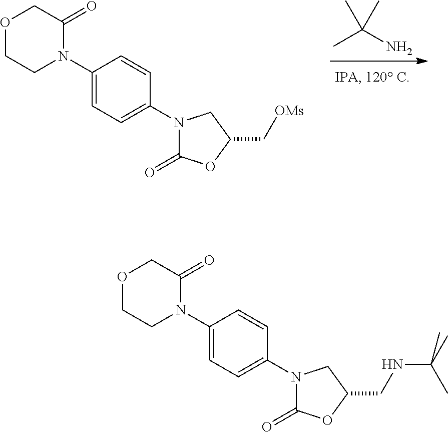

A sealed tube was charged with 4-(4-((R)-5-(methanesulfonyloxymethyl)-2-oxooxazolidin-3-yl)phenyl)morpholin-3-one (383 mg, 1.mmol), isopropanol (IPA) (3.8 mL) and tert-butylamine (0.92 mL, 6 mmol). The mixture was stirred at 120° C. for 12 h. The solvent was evaporated in vacuo. The residue was suspended in EtOAc (5 mL) and treated with NaHCO₃ (5 mL). The aqueous phase was extracted with EtOAc (1×5 mL), the organic phases were combined and dried over MgSO₄ and concentrated in vacuo affording 4-(4-((S)-5-(tert-butylaminomethyl)-2-oxooxazolidin-3-yl)phenyl)morpholin-3-one (298 mg, 83% yield, 91% HPLC-MS) as a brownish solid: ¹H NMR (400 MHz, CDCl₃) δ 7.61 (d, J=8.8 Hz, 2H), 7.35 (d, J=8.8 Hz, 2H), 4.77-4.71 (m, 1H), 4.34 (s, 2H), 4.07-4.02 (m, 3H), 3.91 (dd, J=8.4 Hz, 6.8 Hz, 1H), 3.75 (t, J=4.8 Hz, 2H), 2.94 (dd, J=12.4 Hz, 4.4 Hz, 1H), 2.87 (dd, J=12.4 Hz, 5.2 Hz, 1H), 1.11 (s, 9H).

### Example 8

### Preparation of 4-(4-((S)-5-(tert-butylaminomethyl)-2-oxooxazolidin-3-yl)phenyl)morpholin-3-one (compound III with R₁=tert-butyl) in DMF (by formation of the corresponding methansulfonic salt) (method A)

A mixture of 4-(4-((R)-5-(methanesulfonyloxymethyl)-2-oxooxazolidin-3-yl)phenyl)morpholin-3-one (750 mg, 2 mmol), tert-butylamine (1.2 mL, 8 mmol) in DMF (4 mL) was introduced in a sealed tube and heated at 80° C. for 24 h. The solvent was evaporated by heating in vacuo, the residue was suspended in EtOAc (5 mL) and filtered affording the methanesulfonic salt of 4-(4-((S)-5-(tert-butylaminomethyl)-2-oxooxazolidin-3-yl)phenyl)morpholin-3-one (402 mg, yield 45%, 91% HPLC-MS). This solid was suspended in EtOAc (5 mL) and treated with NaHCO₃ (5 mL). The aqueous phase was extracted with EtOAc (5 mL), the organic phases were combined and dried over MgSO₄ and concentrated in vacuo affording 4-(4-((S)-5-(tert-butylaminomethyl)-2-oxooxazolidin-3-yl)phenyl)morpholin-3-one (227 mg, yield 33%, 97% HPLC-MS) as a brownish solid.

### Example 9

### Crystallization of 4-(4-((S)-5-(tert-butylaminomethyl)-2-oxooxazolidin-3-yl)phenyl)morpholin-3-one (compound III with R₁=tert-butyl) in MIK

4-(4-((S)-5-(tert-Butylaminomethyl)-2-oxooxazolidin-3-yl)phenyl)morpholin-3-one (978 mg, 86% HPLC-MS) was suspended in MIK (4.9 mL, 5 vol) and heated until solution (77° C., bath temperature). The solution was allowed to cool to room temperature (crystallization temperature 30-35° C., bath temperature) and then cooled to 0° C. with an ice batch, filtered, washed with cold MIK (1 vol) and dried under vacuum. 554 mg (yield 57%, 93% HPLC-MS purity) of 4-(4-((S)-5-(tert-butylaminomethyl)-2-oxooxazolidin-3-yl)phenyl)morpholin-3-one were obtained as a brownish solid

### Example 10

### Preparation of 4-(4-((S)-5-(tert-butylaminomethyl)-2-oxooxazolidin-3-yl)phenyl)morpholin-3-one (compound III with R₁=tert-butyl) in DMF (by formation of the corresponding methansulfonic salt) (method B)

To a solution of 4-(4-((S)-5-(tert-butylaminomethyl)-2-oxooxazolidin-3-yl)phenyl)morpholin-3-one (298 mg, 0.86 mmol) (90.6% HPLC-MS) in DMF (2 mL) were added dropwise methanesulfonic acid (50 μL, 0.77 mmol). The resulting mixture was stirred at room temperature during 2 h and evaporated to dryness. CH₂Cl₂ was added and evaporated to dryness (3×3 mL). The resulting solid was suspended in EtOAc (3 mL), stirred, filtered and washed with cold EtOAc (0.5 mL). The resulting solid was treated with EtOAc (3 mL) and NaHCO₃ (3 mL). The layers were separated and the aqueous was extracted with EtOAc (3 mL). The combined organic phases were dried over MgSO₄ and concentrated in vacuo affording 4-(4-((S)-5-(tert-butylaminomethyl)-2-oxooxazolidin-3-yl)phenyl)morpholin-3-one (125 mg, yield 42%). The aqueous phase was evaporated to dryness, dissolved in water (3 mL) and extracted with EtOAc (3×3 mL). The combined organic phases were dried over MgSO₄ and concentrated in vacuo affording 100 mg more. (225 mg, overall yield 75%, 98.4% HPLC-MS) as a brownish solid.

### Example 11

### Preparation of 5-Chloro-N-tert-butyl-N-{[(5S)-2-oxo-3-[4-(3-oxomorpholin-4-yl)phenyl]oxazolidin-5-yl]methyl}thiophene-2-carboxamide (compound II with R₁=tert-butyl)

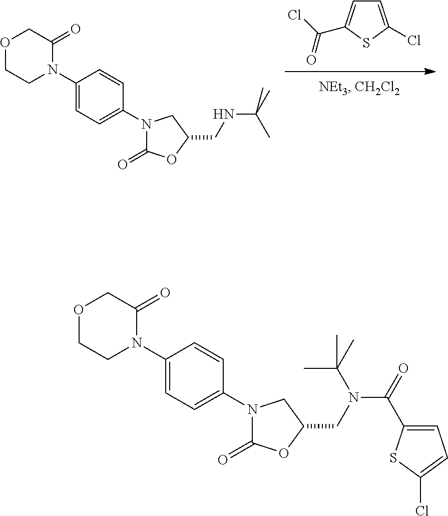

To a solution of 4-(4-((S)-5-(tert-butylaminomethyl)-2-oxooxazolidin-3-yl)phenyl)morpholin-3-one (76 mg, 0.22 mmol) in anhydrous CH₂Cl₂ (0.7 mL) cooled to 0° C. were added NEt₃ (0.15 mL, 1.10 mmol) and, subsequently, 5-chlorothiophene-2-carbonyl chloride (60 mg, 0.33 mmol). The mixture was stirred at room temperature for 24 h, was diluted with CH₂Cl₂ (15 mL) and was washed with H₂O and NaOH 1 M. The organic phase was dried over MgSO₄ and was concentrated in vacuo. The crude obtained was purified by flash column chromatography (CH₂Cl₂/MeOH 98:2, R_{f} 0.20) affording 5-Chloro-N-tert-butyl-N-{[(5S)-2-oxo-3-[4-(3-oxomorpholin-4-yl)phenyl]oxazolidin-5-yl]methyl}thiophene-2-carboxamide (or compound II with R₁=tert-butyl) (55 mg, yield 51%, 90% HPLC-MS) as a white solid. ¹H NMR (400 MHz, CDCl₃) δ 7.47 (d, J=8.8 Hz, 2H), 7.33 (d, J=8.8 Hz, 2H), 7.04 (d, J=4.0 Hz, 1H), 6.81 (d, J=4.0 Hz, 1H), 4.81-4.74 (m, 1H), 4.33 (s, 2H), 4.05-3.97 (m, 5H), 3.74 (t, J=5.2 Hz, 2H), 3.55 (dd, J=9.2 Hz, 6.4 Hz, 1H), 1.54 (s, 9H).

### Example 12

### Preparation of rivaroxaban by dealkylation of 5-Chloro-N-tert-butyl-N-{[(5S)-2-oxo-3-[4-(3-oxomorpholin-4-yl)phenyl]oxazolidin-5-yl]methyl}thiophene-2-carboxamide using ethyl acetate and hydrochloric acid

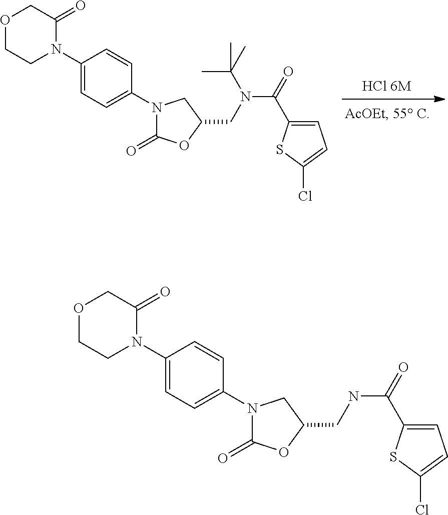

To a suspension of 5-chloro-N-tert-butyl-N-({(5S)-2-oxo-3-[4-(3-morpholin-4-yl)phenyl]-1,3-oxazolidin-5-yl}methyl)thiophene-2-carboxamide (99 mg, 0.20 mmol) in EtOAc (0.6 mL) was added 6 M HCl solution (74 μL, 0.4 mmol). The mixture was heated to 55° C. for 2 h, was cooled to room temperature and was basified with 6 M NaOH to approximately pH 9. The solvent was evaporated and the residue was diluted with H₂O and extracted with CH₂Cl₂. The organic phases were dried over MgSO₄ and concentrated in vacuo, affording 5-Chloro-N-{[(5S)-2-oxo-3-[4-(3-oxomorpholin-4-yl)phenyl]oxazolidin-5-yl]methyl}thiophene-2-carboxamide (rivaroxaban or compound I) (79 mg, yield 90%, 92% HPLC-MS) as a white solid.

### Example 13

### Preparation of rivaroxaban by one pot process (from 4-(4-((S)-5-(tert-butylaminomethyl)-2-oxooxazolidin-3-yl)phenyl)morpholin-3-one (compound III with R₁=tert-butyl))

To a suspension of 4-(4-((S)-5-(tert-butylaminomethyl)-2-oxooxazolidin-3-yl)phenyl)morpholin-3-one (554 mg, 1.59 mmol) (92.6% HPLC-MS) in toluene (2.1 mL) were added NEt₃ (244 μL, 1.73 mmol) and, subsequently, a solution of 5-chlorothiophene-2-carbonyl chloride (3.46 mL of a solution 0.69 M in toluene). The mixture was stirred at 120° C. for 2 h, then was allowed to cool to 55° C. and 6 M HCl solution was added (530 μL). The mixture was stirred at 55° C. for 4 h, was cooled to room temperature and was basified with 3 M NaOH to pH 9. The solid was filtered and washed with H₂O (0.5 mL) affording 5-Chloro-N-{[(5S)-2-oxo-3-[4-(3-oxomorpholin-4-yl)phenyl]oxazolidin-5-yl]methyl}thiophene-2-carboxamide (rivaroxaban or compound I) (554 mg, yield 81%, 93.7% HPLC-MS) as a brownish solid.

### Example 14

### Purification of rivaroxaban by crystallization in EtOH/H₂O

To a round-bottom flask equipped with magnetic stirrer containing rivaroxaban hydrate (199.5 mg, 0.44 mmol, 95.0% HPLC) was added ethanol (7.2 mL). The resultant suspension was heated at 70° C. and water was added dropwise (3.1 mL). This mixture was slowly cooled to room temperature and then stirred for 1.5 hours. The precipitated solid was filtered with a sintered funnel (porosity 3) and dried under vacuum at room temperature to give rivaroxaban as a white solid (152.4 mg, 80% yield, 97.3% HPLC).

### Example 15

### Purification of rivaroxaban by crystallization in dioxane

To round-bottom flask equipped with magnetic stirrer containing rivaroxaban hydrate (250 mg, 0.55 mmol, 92.0% HPLC) was added dioxane (18.75 mL). The resultant suspension was heated at reflux and the remaining solid was filtered with a sintered funnel (porosity 4). The resulting solution was cooled to room temperature and then stirred for 3.5 hours. The precipitated solid was filtered with a sintered funnel (porosity 3) and dried under vacuum at room temperature to give rivaroxaban as a light yellow solid (177.6 mg, 74% yield, 98.2% HPLC).

### Example 16

### Preparation of 4-(4-((S)-5-(tert-octylaminomethyl)-2-oxooxazolidin-3-yl)phenyl)morpholin-3-one (compound III with R₁=tert-octyl)

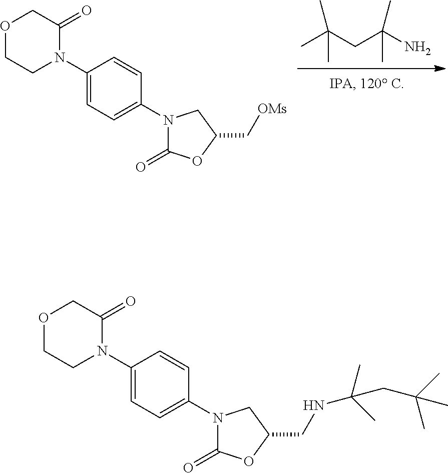

To a suspension of 4-(4-((R)-5-(methanesulfonyloxymethyl)-2-oxooxazolidin-3-yl)phenyl)morpholin-3-one (372 mg, 1 mmol) in isopropanol (IPA) (1.7 mL) was added tert-octylamine (0.418 mL, 2.5 mmol). The mixture was stirred at 120° C. for 5 days, the yellow solution obtained was cooled to 0° C. and was stirred for a further 1 h at 0° C. The resulting brownish precipitate was isolated by filtration, washed with cold IPA, and dried affording 4-(4-((S)-5-(tert-octylaminomethyl)-2-oxooxazolidin-3-yl)phenyl)morpholin-3-one (or compound III with R₁=tert-octyl) (321 mg, yield 80%, 80% HPLC-MS) as a pale brown solid. ¹H NMR (400 MHz, CDCl₃) δ 7.60 (d, J=8.8 Hz, 2H), 7.35 (d, J=8.8 Hz, 2H), 4.81-4.68 (m, 1H), 4.34 (s, 2H), 4.10-4.00 (m, 3H), 3.93 (dd, J=8.4 Hz, 6.8 Hz, 1H), 3.75 (t, J=4.8 Hz, 2H), 2.97 (dd, J=12.4 Hz, 4.4 Hz, 1H), 2.86 (dd, J=12.4 Hz, 5.2 Hz, 1H), 1.15 (s, 6H), 1.04 (s, 2H), 0.99 (s, 9H).

### Example 17

### Preparation of 5-Chloro-N-tert-octyl-N-{[(5S)-2-oxo-3-[4-(3-oxomorpholin-4-yl)phenyl]oxazolidin-5-yl]methyl}thiophene-2-carboxamide (compound of formula II with R1=tert-octyl)

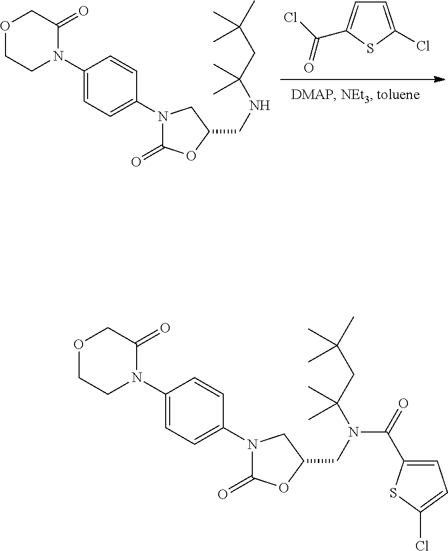

Under Ar, 5-chlorothiophene-2-carboxylic acid (150 mg, 0.92 mmol) was dissolved in AcOEt (5 mL). The solution was cooled to 0° C. and anhydrous DMF (1 drop) and subsequently oxalyl chloride (0.10 mL, 1.14 mmol) were added. The solution was stirred at 0° C. for 2 h and the mixture was then concentrated on a rotary evaporator at room temperature. The purity of the brown liquid (186 mg, 111% of the theoretical mass) was checked by ¹H NMR, showing the complete conversion into the acid chloride, along with remaining DMF. ¹H NMR (200 MHz, CDCl₃) δ 7.80 (d, J=4.2 Hz, 2H), 7.04 (d, J=4.2 Hz, 2H).

The acid chloride was dissolved in toluene (1.0 mL) and dropwise transferred under Ar onto a solution of 4-(4-((S)-5-((tert-octylamino)methyl)-2-oxooxazolidin-3-yl)phenyl)morpholin-3-one (150 mg, 0.37 mmol), 4-dimethylaminopyridine (5 mg, 0.041 mmol) and Et₃N (0.25 ml, 1.79 mmol) in toluene (1.0 mL). The mixture was heated to reflux for 10 h, cooled, diluted with CH₂Cl₂ (10 mL) and washed with H₂O (10 mL) and NaOH 1N (10 mL). The organic phase was dried over anhydrous Na₂SO₄ and evaporated in vacuo. The crude obtained was purified by flash chromatography (CH₂Cl₂:MeOH 95:5, R_{f} 0.27) affording 5-Chloro-N-tert-octyl-N-{[(5S)-2-oxo-3-[4-(3-oxomorpholin-4-yl)phenyl]oxazolidin-5-yl]methyl}thiophene-2-carboxamide (or compound II with R1=tert-octyl) (112 mg, yield 55%, 89% HPLC-MS) as an off-white solid. ¹H NMR (400 MHz, CD₃OD) δ 7.51 (d, J=8.8 Hz, 2H), 7.36 (d, J=8.8 Hz, 2H), 7.12 (d, J=4.0 Hz, 1H), 6.90 (d, J=4.0 Hz, 1H), 4.98-4.91 (m, 1H), 4.27 (s, 2H), 4.25 (dd, J=16.8, 10.0, 1H), 4.15 (t, J=10.0, 1H), 4.04-4.02 (m, 2H), 3.97 (dd, J=16.8, 3.2, 1H), 3.79-3.74 (m, 2H), 3.59 (dd, J=9.2, 6.0, 1H), 2.48 (d, J=14.8, 1H), 1.84 (d, J=14.8, 1H), 1.64 (s, 6H), 1.05 (s, 9H).

### Example 18

### Preparation of rivaroxaban by dealkylation of 5-Chloro-N-tert-octyl-N-{[(5S)-2-oxo-3-[4-(3-oxomorpholin-4-yl)phenyl]oxazolidin-5-yl]methyl}thiophene-2-carboxamide using hydrochloric acid and tetrahydrofuran

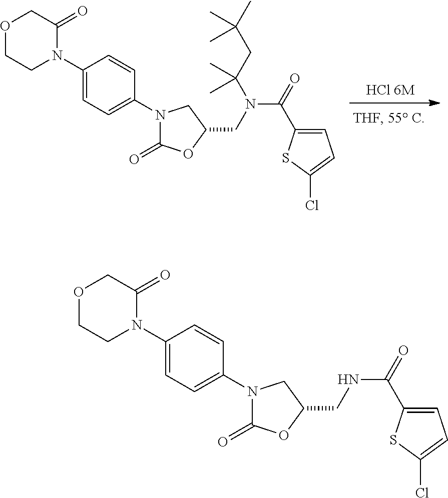

To a solution of 5-Chloro-N-tert-octyl-N-{[(5S)-2-oxo-3-[4-(3-oxomorpholin-4-yl)phenyl]oxazolidin-5-yl]methyl}thiophene-2-carboxamide (37 mg, 0.068 mmol) in THF (1.0 mL), HCl 6 M (0.050 mL, 0.30 mmol) was added. The solution was heated to 55° C. for 2 hours and 15 minutes, cooled, dried over anhydrous Na₂SO₄ and evaporated in vacuo. The crude was purified by flash chromatography (CH₂Cl₂:MeOH 95:5, R_{f} 0.17) affording 5-Chloro-N-{[(5S)-2-oxo-3-[4-(3-oxomorpholin-4-yl)phenyl]oxazolidin-5-yl]methyl}thiophene-2-carboxamide (rivaroxaban or compound of formula I) (10 mg, yield 34%, 100% HPLC-MS, [α]_{D} −38.9 (c=1, DMSO). Ref. Roehring et al., Journal of Medicinal Chemistry, 2005, Vol. 48, pp 5900, [α]_{D} −38 (c=0.3, DMSO)) as a white solid. ¹H NMR (d₆-DMSO, 400 MHz) δ 8.95 (t, J=5.6, 1H), 7.68 (d, J=4.0, 1H), 7.54 (d, J=8.8, 2H), 7.39 (d, J=8.8, 2H), 7.18 (d, J=4.0, 1H), 4.86-4.80 (m, 1H), 4.20-4.16 (m, 1H), 4.18 (s, 2H), 3.97-3.94 (m, 2H), 3.84 (dd, J=9.2, 6.0, 1H), 3.71-3.69 (m, 2H), 3.59 (t, J=5.6, 2H).

### REFERENCES CITED IN THE DESCRIPTION

EP 1261606.
S. J. Brickner et al., J. Med. Chem.—1996, Vol. 39, pp. 673.
WO 2003/2556.
EP 1114819.
Roehring et al., Journal of Medicinal Chemistry—2005, Vol. 48, pp. 5900.
DE 10342570.
WO 2005/26135.
WO 2006/116713.

1. A process for the preparation of rivaroxaban of formula (I), or a pharmaceutically acceptable salt thereof, 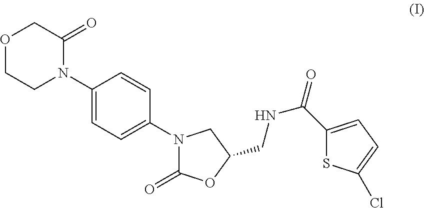
comprising submitting a compound of formula (III) 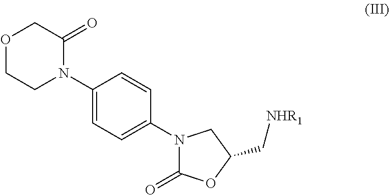
wherein R₁ is a (C₄-C₁₀)-alkyl radical which is attached to the N atom by a tertiary C atom;
first to an acylation reaction with a compound of formula (IV) 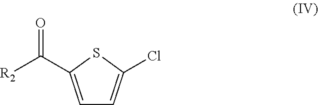
wherein R₂ is a radical selected from the group consisting of a halogen and a radical of formula (C₁-C₈)COO;
and then to a dealkylation reaction; and, optionally, treating the compound (I) thus obtained with a pharmaceutically acceptable acid to form the corresponding salt.

2. The process according to claim 1, wherein the acylation reaction and the dealkylation reaction are performed in one pot.

3. The process according to claim 1, wherein R₁ is selected from the group consisting of tert-butyl and tert-octyl.

4. The process according to claim 1, wherein the acylation reaction is carried out with a compound of formula (IV) wherein R₂ is chloride.

5. The process according to claim 1, wherein a compound of formula (V), 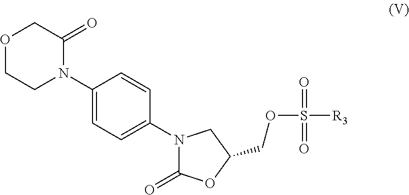
wherein R₃ is a radical selected from the group consisting of (C₁-C₄)-alkyl, phenyl, and phenyl mono- or disubstituted by a (C₁-C₄)-alkyl radical,
is submitted to an amination reaction with an amine of formula R₁NH₂ where R₁ is a (C₄-C₁₀)-alkyl radical which is attached to the N atom by a tertiary C atom, to give the compound of formula (III) as defined in claim 1.

6. A process for the preparation of a compound of formula (III), or a pharmaceutically acceptable salt thereof, 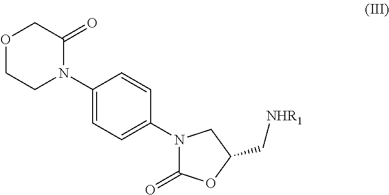
wherein R₁ is as defined in claim 1;
comprising submitting a compound of formula (V), 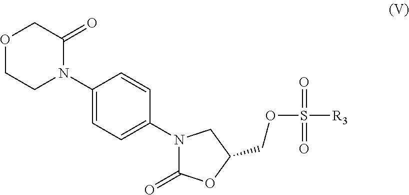
wherein R₃ is a radical selected from the group consisting of (C₁-C₄)-alkyl, phenyl, and phenyl mono- or disubstituted by a (C₁-C₄)-alkyl radical,
to an amination reaction with an amine of formula R₁NH₂ where R₁ is a (C₄-C₁₀)-alkyl radical which is attached to the N atom by a tertiary C atom.

7. The process according to claim 6, wherein R₃ is methyl.

8. The process according to claim 6, wherein the amine used is selected from the group consisting of, tert-butylamine, and tert-octylamine.

9. A compound of formula (III), or a pharmaceutically acceptable salt thereof, 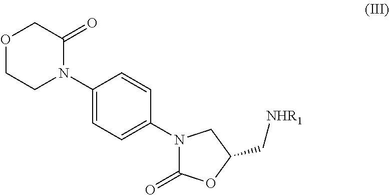
wherein R₁ is a (C₄-C₁₀)-alkyl radical which is attached to the N atom by a tertiary C atom.

10. The compound according to claim 9, wherein the salts are selected from the group consisting of sulfonate, hydrochloride, hydrobromide, and tosylate salts.

11. The compound according to claim 9, wherein the R₁ is a tert-(C₄-C₈)alkyl.

12. The compound according to claim 11, wherein R₁ is selected from the group consisting of tert-butyl, and tert-octyl.

13. A compound of formula (II) 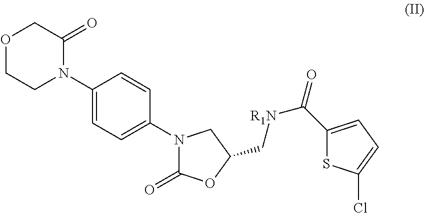
wherein R₁ is a (C₄-C₁₀)-alkyl radical which is attached to the N atom by a tertiary C atom.

14. The compound according to claim 13, wherein the R₁ is a tert-(C₄-C₈)alkyl.

15. The compound according to claim 14, wherein R₁ is selected from the group consisting of tert-butyl, and tert-octyl.

16. The process according to claim 2, wherein R₁ is selected from the group consisting of tert-butyl and tert-octyl.

17. The process according to claim 7, wherein the amine used is selected from the group consisting of tert-butylamine, and tert-octylamine.
